(19) United States
(12) Patent Application Publication
(10) Pub. No.: US 2009/0304685 A1
(43) Pub. Date: Dec. 10, 2009

(54) Anti-Factor Xlla Therapy

(76) Inventor: David J. Pritchard, Scone (GB)

Correspondence Address: Leon R Yankwich;Yankwich & Associates, 201 Broadway, Cambridge, MA, 02139, US

(21) Appl. No.: 12/226,256

(22) PCT Filed: Apr. 12, 2007
(86) PCT No.: PCT/GB2007/001351

§ 371 Date: Jul. 20, 2009

(30) Foreign Application Priority Data
Apr. 13, 2006 (GB) 0607515.4

(51) Int. Cl. A61K 39/395; C07K 16/40; C12Q 1/37

(52) U.S. Cl. 424/133.1; 424/158.1; 424/146.1; 530/389.8; 530/388.26; 530/387.3; 530/391.3; 435/23

(57) ABSTRACT

A method is disclosed for preventing arterial thrombosis in a subject comprising administering to said subject a therapeutically effective amount of an antibody or epitope-binding fragment or derivative thereof, wherein said antibody, fragment or derivative specifically binds to activated Factor XIIa and prevents the interaction of activated Factor XIIa with its physiological substrates.

### INTRODUCTION

The present invention relates to methods of preventing arterial thromboses and to related products, diagnostic methods, imaging methods and drug targeting methods.

### BACKGROUND OF THE INVENTION

Arterial thrombosis is a major cause of illness and death. An occlusive or near-occlusive thrombus in the cardiac arteries can result in cessation of most of the blood supply to part of the heart leading to ischaemia and myocardial infarction. A thrombus that results in a less dramatic reduction in blood supply to the heart can lead to angina. Thrombosis of arteries leading to ischaemia of brain tissue is the most common cause of stroke. It has been demonstrated that Factor XII is necessary for arterial thrombus formation in mammals (Renne et al. JEM, 202, 271-281 (2005)). WO2006/066878 discloses the use of at least one antibody and/or one inhibitor for inhibiting factor XII and preventing the formation and/or the stabilization of thrombi and/or thrombus growth.

The subject of the present invention is, in a general aspect the prevention of the formation and/or the stabilization of thrombi by the use of antibodies directed against activated Factor XII (whereas WO2006/066878 describes the use of antibodies directed against the Factor XII zymogen).

There is considerable evidence that deficiency of Factor XII does not contribute to a bleeding diathesis, hence inhibition of activated Factor XII is an attractive therapeutic approach for the prevention of arterial thrombosis, as whilst it may inhibit thrombosis formation, there will not be an associated risk of bleeding as observed with a number of other anti-thrombotic agents.

Factor XII is an inactive zymogen present in normal blood. It is readily converted, in vitro, in the presence of kallikrein, high molecular weight kininogen and a negatively charged surface into a form of Factor XII known as Factor XIIa, that is enzymatically active. In vitro, two forms of XIIa have been reported. The 80 Kd form of the serine proteinase, often called Factor αXIIa, has a 52 Kd heavy chain linked by a disulphide bond to a 28 Kd light chain. Proteolysis of this factor releases a peptide from the heavy chain, and results in a product, Factor βXIIa, that retains serine protease activity, but in which the 28 Kd chain of Factor αXIIa is disulphide-linked to a small peptide fragment derived from the former 52-Kd heavy chain. In many cases the small peptide fragment has a molecular weight of about 1000 d, but fragments of different size have been observed in vitro. A further form of activated Factor XIIa found in-vivo and having a molecular weight of 53 Kd was first reported in PCT/GB2006/000072, the disclosure of which is incorporated herein by reference.

WO 90/08835 discloses an immunoassay for Factor XIIa. WO 90/08835 also discloses monoclonal antibodies 2/215 and 201/9, which bind to all known molecular weight forms of activated Factor XIIa, and methods for their production. Monoclonal antibody (mAb) 2/215 is produced by hybridoma 2/215, deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606 and redeposited at ECACC on 14 Jun. 2004 under the deposit number 04061403. Hybridoma 201/9, producing monoclonal antibody 201/9, was deposited at ECACC on 18 Jan. 1990 under deposit number 90011893 and redeposited at ECACC on 14 Jun. 2004 under deposit number 04061402.

Factor XIIa has long been known to be involved in the contact system of blood coagulation in vivo. More recent work indicates that Factor XIIa is also involved in other systems, including fibrinolysis, kininogensis, and also complement activation and angiogenesis. Many clinical and experimental data are accumulating to suggest that the contact system extends beyond haemocoagulation and that it has a role in maintaining vascular wholeness and blood pressure, that it influences various functions of endothelial cells, and that it is involved in control of fibrinolysis and in maintaining the constitutive anticoagulant character of the intravascular space. Further clinical and experimental studies indicate that the contact system is involved in acute and chronic inflammation, shock of different aetiologies, diabetes, allergy, thrombo-haemorrhagic disorders including disseminated intravascular blood coagulation, and oncological diseases. Such conditions, include sepsis, spontaneous abortion and thromboembolism. In addition, Factor XIIa may be involved in tissue defence and repair. Yarovaya et al. (Yarovaya, G. A., Blokhina, T. B. & Neshkova, E. A. Contact system. New concepts on activation mechanisms and bioregulatory functions. Biochemistry (Mosc). 2002 January; 67(1):13-24) is a recent review of the contact system and new concepts on activation mechanisms and bioregulatory functions.

WO 04/057343 discloses that Factor XIIa exists in a variety of forms in the body and that measurement of levels of those different forms provides valuable information relating to a variety of clinical conditions. WO 91/17258 (Inventor Nuijens et al) claims an antibody that binds to Factor XII/activated Factor XII used as a therapeutic in sepsis. WO 9936439 (Inventor Seale et al) claims a polypeptide, derivable from a Haementeria leech that can be used as a therapeutic for a number of conditions related to Factor XII.

### SUMMARY OF THE INVENTION

The invention provides a method of preventing arterial thrombosis in a subject comprising administering to said subject a therapeutically effective amount of an antibody or epitope-binding fragment or derivative thereof wherein said antibody, fragment or derivative specifically binds to activated Factor XIIa and prevents the interaction of activated Factor XIIa with its physiological substrates.

The invention also provides an antibody or epitope-binding fragment or derivative thereof, wherein said antibody specifically binds to activated Factor XIIa and prevents the interaction of activated Factor XIIa with its physiological substrates for use as a medicament.

The invention also provides a pharmaceutical composition comprising an antibody or epitope-binding fragment or derivative thereof according to the invention together with a pharmaceutically acceptable carrier.

The invention also provides use of an antibody or epitope-binding fragment or derivative thereof, wherein said antibody specifically interacts with activated Factor XIIa and prevents the interaction of activated Factor XIIa with its physiological substrates in the manufacture of a medicament for preventing arterial thrombosis in a subject.

The invention also provides an antibody or epitope-binding fragment or derivative thereof, wherein said antibody specifically interacts with activated Factor XIIa and prevents the interaction of activated Factor XIIa with its physiological substrates for use as a medicament for preventing arterial thrombosis in a subject

The invention also provides a method of predicting the risk of subsequent restenosis in a subject elected to undergo percutaneous coronary intervention comprising measuring the level of activated Factor XIIa in the blood of said subject and assigning said subject to a high risk group if the subject's plasma concentration of activated Factor XIIa is significantly different to that of a reference population, or if the subject's plasma concentration of activated Factor XIIa increases by a factor significantly different to that of a reference population following administration to said subject of heparin and contrast agent in preparation for angioplasty.

The invention also provides an in vivo imaging agent comprising an antibody or epitope-binding fragment or derivative thereof according to the invention attached to a marker moiety.

The invention also provides a method of imaging sites of thrombus formation in a subject comprising administering to said subject of an in vivo imaging agent according to the invention followed by the detection of the imaging agent marker moiety in vivo.

The invention also provides a therapeutic agent comprising an antibody or epitope-binding fragment or derivate thereof according to the invention attached to a therapeutic compound.

The invention also provides a method of treating a disease characterised by undesirable thrombus formation in a subject comprising administering to said subject a therapeutic agent according to the invention.

The invention also provides an antibody or epitope-binding fragment or derivative thereof against non-activated Factor XII for use in preventing arterial thrombosis in a subject wherein said antibody or epitope-binding fragment or derivative thereof prevents multi molecular assemblies of Factor XII or wherein said antibody or epitope-binding fragment or derivative thereof prevents formation of activated Factor XIIa.

The invention also provides a method of preventing arterial thrombosis in a subject comprising administering to said subject a therapeutically effective amount of an antibody or epitope-binding fragment or derivative thereof, wherein said antibody or epitope-binding fragment or derivative thereof prevents multi molecular assemblies of Factor XII or wherein said antibody or epitope-binding fragment or derivative thereof prevents formation of activated Factor XIIa.

### BRIEF DESCRIPTION OF THE FIGURES

FIG. 1 shows levels of plasma Factor XIIa in eleven patients undergoing elective PCI.

FIG. 2 shows 12-month survival data related to quartile for plasma Factor XIIa level for 871 patients admitted to hospital with chest pain.

FIG. 3 shows 12-month survival data related to quartile for plasma Factor XIIa level for a subgroup of the patients shown in FIG. 2, those admitted to hospital with chest pain and with plasma TnT>0.05 ng/ml.

FIG. 4 shows 12-month survival data related to quartile for plasma Factor XIIa level for a subgroup of the patients shown in FIG. 2, those admitted to hospital with chest pain and with plasma Tnt≦0.05 ng/ml.

FIG. 5 shows changes observed in plasma Factor XIIa concentration in a group of patients four days following admission to hospital with myocardial infarction.

FIG. 6 shows the same data as shown in FIG. 5 presented as percentage change in plasma Factor XIIa concentration.

### DEFINITIONS

Factor XIIa, also called activated Factor XII, denotes any enzymatically active form or fragment of the zymogen, Factor XII, including Factor αXIIa, Factor βXIIa and 53 Kd Factor XIIa.

Monoclonal antibody (mAb) 2/215, also called antibody 2/215, is the antibody produced by hybridoma 2/215, deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606, and redeposited at ECACC on 14 Jun. 2004 under the deposit number 04061403.

Monoclonal antibody (mAb) 2/215 analogue denotes an antibody that has Factor XIIa binding properties that are substantially the same as those of mAb 2/215.

Monoclonal antibody (mAb) 201/9, also called antibody 201/9, is the antibody produced by hybridoma 201/9, which was deposited at ECACC on 18 Jan. 1990 under deposit number 90012512 and redeposited at ECACC on 14 Jun. 2004 under the deposit number 04031402.

Monoclonal antibody (mAb) 201/9 analogue denotes an antibody that has Factor XIIa binding properties that are substantially the same as those of mAb 201/9.

### DETAILED DESCRIPTION OF THE INVENTION

The invention is based on the realisation that the use of antibodies directed against the activated Factor XII as opposed to Factor XII zymogen (as described in WO2006/066878) confers a number of advantages. Factor XII zymogen circulates throughout the body a relatively high concentration (around 40 mg/litre) whereas the systemic concentration of activated Factor XII is around 2 ug/litre, thus the concentration of Factor XII is around 20,000 times that of activated Factor XII and therefore very much lower doses of antibody could be used in the latter case. Additionally, as demonstrated in this document, high concentrations of activated Factor XII are evident within the thrombus itself, thus administration of antibodies to activated Factor XII will target and localize at the pathogenic site of thrombus formation, whereas antibodies that show significant reactivity with the Factor XII zymogen will tend to be distributed systemically. Another advantage of using antibodies directed against activated Factor XII is that it has been demonstrated that activated Factor XII exists in a number of different forms (WO 04/057343 and PCT/GB2006/000072). Antibodies directed against activated Factor XII will react with these different forms, whereas antibodies raised against Factor XII zymogen may not react with activated forms as the relevant epitopes are missing. Additionally, data from clinical trials suggests that the level of activated Factor XII is an important contributory factor for thrombotic risk, whereas no such association was found between Factor XII zymogen and thrombotic risk, thus indicating that the activated Factor XII is the clinically important parameter. Further evidence that activated Factor XII is the clinically relevant parameter, and therefore a better therapeutic target than Factor XII zymogen is provided by the observation that decreases in activated Factor XII results in an improved prognosis, whereas increases in activated Factor XII result in a poorer prognosis.

The invention provides a method of preventing arterial thrombosis in a subject comprising administering to said subject a therapeutically effective amount of an antibody or epitope-binding fragment or derivative thereof, wherein said antibody, fragment or derivative specifically binds to activated Factor XIIa and prevents the interaction of activated Factor XIIa with its physiological substrates.

Preferably, said antibody or epitope-binding fragment or derivative thereof binds to Factor αXIIa or to Factor βXIIa or to 53 Kd factor XIIa, and has a corrected cross-reactivity with un-activated Factor XII of 0.1% or less.

According to certain embodiments of the invention, said administration is prior to, during or following angioplasty carried out on said subject. Administration following angioplasty would be in order to reduce the likelihood of subsequent undesirable thrombus formation.

According to an alternative embodiment, said administration is following myocardial infarction in said subject. Administration following myocardial infarction would be in order to reduce the likelihood of further undesirable thrombus formation.

According to certain embodiments, said administration is to a subject having an estimated risk for Coronary heart disease of more than 10% preferably of more than 12, 14, 16, 18, 20, 22, 24, 26, 28 or 30% as defined using the Framingham risk scoring method. Said administration would be in order to reduce the risk of undesirable thrombus formation and therefore the risk of coronary heart disease.

According to certain embodiments, said administration is to a subject having a plasma concentration of activated Factor XIIa of significantly different to that of a reference population as measured before administration of said antibody or epitope-binding fragment or derivative thereof.

According to certain embodiments, said administration is to a subject's plasma concentration of activated Factor XIIa increases by a factor significantly different to that of a reference population following administration to said subject of heparin and contrast agent in preparation for angioplasty. Subjects in this group of angioplasty patients have been found to be at a particularly high risk of restenosis following angioplasty.

As an example, a plasma concentration of activated Factor XIIa of more than 150 μM may be significantly different to that of a reference population. An increase in plasma concentration of activated Factor XIIa of a factor of 2 or more may be regarded as significantly different to that of a reference population.

Said antibody or epitope-binding fragment or derivative thereof may be a monoclonal antibody or epitope-binding fragment or derivative thereof.

Preferably, said antibody or epitope-binding fragment or derivative thereof is mAb 2/215 or an analogue thereof or mAb 201/9 or an analogue thereof or an epitope-binding fragment or derivative of mAb 2/215 or an analogue thereof or of mAb 201/9 or an analogue thereof.

Said antibody or epitope-binding fragment or derivative thereof may be a Fab fragment or a (Fab′)₂ fragment.

Preferably, said antibody or epitope-binding fragment or derivative thereof is a humanised antibody or epitope-binding fragment or derivative thereof.

The invention also provides an antibody or epitope-binding fragment or derivative thereof, wherein said antibody specifically binds to activated Factor XIIa and prevents the interaction of activated Factor XIIa with its physiological substrates for use as a medicament.

Such an antibody or epitope-binding fragment or derivative thereof may incorporate one or more additional features described above in respect of a method of the invention.

The invention also provides a pharmaceutical composition comprising an antibody or epitope-binding fragment or derivative thereof according to the invention together with a pharmaceutically acceptable carrier.

The invention also provides use of an antibody or epitope-binding fragment or derivative thereof wherein said antibody specifically interacts with activated Factor XIIa and prevents the interaction of activated Factor XIIa with its physiological substrates in the manufacture of a medicament for preventing arterial thrombosis in a subject.

The invention also provides a method of predicting the risk of subsequent restenosis in a subject elected to undergo percutaneous coronary intervention comprising measuring the level of activated Factor XIIa in the blood of said subject and assigning said subject to a high risk group if the subject's plasma concentration of activated Factor XIIa is significantly different to that of a reference population or if the subject's plasma concentration of activated Factor XIIa increases by a factor significantly different to that of a reference population following administration to said subject of heparin and contrast agent in preparation for angioplasty.

As an example, a plasma concentration of activated Factor XIIa of more than 150 μM may be significantly different to that of a reference population. An increase in plasma concentration of activated Factor XIIa of a factor of 2 or more may be regarded as significantly different to that of a reference population.

A subject placed in a high risk group may be given extra treatment or monitoring to prevent restenosis. Alternatively, the elected PCI may be abandoned.

The invention also provides an in vivo imaging agent comprising an antibody or epitope-binding fragment or derivative thereof according to the invention attached to a marker moiety.

The invention also provides a method of imaging site of thrombus formation in a subject comprising administration to said subject of an in vivo imaging agent according to the invention followed by the detection of the imaging agent marker moiety in vivo.

The invention also provides a therapeutic agent comprising an antibody or epitope-binding fragment or derivative thereof according to the invention attached to a therapeutic compound.

Said antibody or epitope-binding fragment or derivative thereof, and said subject may be as defined above in respect of a method of the invention, said medicament may be for the treatment of a disease associated with undesirable thrombosis function. For example, said medicament may be for administration following angioplasty, myocardial infarction, or stroke.

The invention also provides an antibody or epitope-binding fragment or derivative thereof according to the invention, wherein said antibody specifically interacts with activated Factor XIIa and prevents the interaction of activated Factor XIIa with its physiological substrates for use as a medicament for preventing arterial thrombosis in a subject

Said therapeutic compound is preferably a thrombolytic agent or other anti-thrombotic agent, for example, streptokinase, urokinase, tissue plasminogen activator (tPA), Tirofiban, Clopidogrel, or Tenecteplase.

The invention also provides a method of treating a disease characterised by undesirable thrombus formation in a subject comprising administering to said subject a therapeutic agent according to the invention.

The invention also provides an antibody or epitope-binding fragment or derivative thereof against non-activated Factor XII for use in preventing arterial thrombosis in a subject wherein said antibody or epitope-binding fragment or derivative thereof prevents multi molecular assemblies of Factor XII or wherein said antibody or epitope-binding fragment or derivative thereof prevents formation of activated Factor XIIa.

The invention also provides a method of preventing arterial thrombosis in a subject comprising administering to said subject a therapeutically effective amount of an antibody or epitope-binding fragment or derivative thereof, wherein said antibody or epitope-binding fragment or derivative thereof prevents multi molecular assemblies of Factor XII or wherein said antibody or epitope-binding fragment or derivative thereof prevents formation of activated Factor XIIa.

### Production of Activated Factor XIIa-Specific Antibodies

For the production of activated Factor XIIa-specific antibodies for use in the invention, various host animals may be immunized by injection with a suitable antigen (see below for details of antigen selection). Such host animals may include but are not limited to pigs, rabbits, mice, goats, horses and rats. Various adjuvants may be used to increase the immunological response, depending on the host species, including but not limited to Freund's adjuvant (complete and incomplete), mineral salts such as aluminium hydroxide or aluminium phosphate, surface active substances such as lysolecithin, pluronic polyols, polyanions, peptides, oil emulsions, and potentially useful human adjuvants such as BCG (bacille Calmette-Guerin) and Corynebacterium parvum.

Alternatively, the immune response may be enhanced by combination and/or coupling with molecules of response-enhancing agents, for example, keyhole limpet haemocyanin, tetanus toxoid, diphtheria toxoid, ovalbumin, cholera toxin or fragments thereof.

Polyclonal antibodies may also be used in the invention. Polyclonal antibodies are heterogeneous populations of antibody molecules derived from sera of the immunized animals.

Monoclonal antibodies, which are homogeneous populations of antibodies to a particular antigen, can be obtained by any technique that provides for the production of antibody molecules by continuous cell lines in culture.

These include, but are not limited to, the hybridoma technique of Kohler and Milstein, (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 7796).

A hybridoma that produces a mAb according to the present invention may be cultivated in vitro or in vivo and the resulting mAb purified by conventional techniques. Production of high titres of mAbs in vivo, may make this a preferred method of production. However, in vitro production may be preferred where legal, commercial or ethical constrains regarding the use of animals make in vivo production undesirable.

### Humanized Antibodies

In addition, techniques developed for the production of “chimeric antibodies” and in particular “humanized antibodies” (Morrison et al., 1984, Proc. Natl. Acad. Sci., 81: 6851-6855; Neuberger et al., 1984, Nature, 312: 604-608; Takeda et al., 1985, Nature, 314: 452-454) by splicing the genes from a mouse antibody molecule of appropriate antigen specificity together with genes from an antibody molecule of a different species, for example, a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,075,181 and 5,877,397 and their respective disclosures which are herein incorporated by reference in their entirety. Also encompassed by the present invention is the use of fully humanized monoclonal antibodies as described in U.S. Pat. No. 6,150,584 which is herein incorporated by reference in their entirety. Human or humanised animal mAbs may be preferable for therapeutic use in humans.

### Antibody Derivatives

An example of an antibody derivative for use in the invention is a single chain antibody. Techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 341: 544-546) can be adapted to produce single chain antibodies. Single chain antibodies are formed by linking the heavy and light chain fragments of the Fv region via an amino acid bridge, resulting in a single chain polypeptide.

### Antibody Fragments

Antibody fragments that recognize specific epitopes may be generated by known techniques. For example, such fragments include, but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246: 1275-1281) to allow rapid and easy identification of monoclonal Fab fragments with the desired specificity.

Antibodies of the invention and fragments and derivatives thereof may be of any immunoglobulin class including IgG, IgM, IgE, IgA, IgD and any subclass thereof.

### Selection and Preparation of Suitable Antigens for Production of Antibodies

### Antigen Selection

Antibodies according to the invention are required to bind to at least one form of activated Factor XIIa in preference to non-activated Factor XII. They should therefore recognise an epitope or epitopes that are present and accessible on activated XIIa but are absent or inaccessible on non-activated Factor XII. One approach to antigen selection is therefore to select a peptide antigen having an amino acid sequence that is accessible on Factor αXIIa, Factor βXIIa or 53 Kd Factor XIIa but inaccessible in non-activated Factor XIIa.

### Antigen Preparation

The size, extent of aggregation and relative nativity, that is to say, the relative lack or denaturation, of protein antigens can all dramatically affect the quality and quantity of antibody produced. Small polypeptides (<10 Kda) and non-protein antigens generally need to be conjugated or cross-linked to larger, immunogenic, carrier proteins to increase immunogenicity and provide T cell epitopes. Injection of soluble, non-aggregated proteins may induce tolerance rather than a satisfactory antibody response. It may therefore be desirable to conjugate the antigen to a larger protein such as keyhole limpet haemocyanin (KLH) or bovine serum albumen (BSA) Poly-L-lysine has also been used successfully as a backbone for small antigenic peptides.

Antigens should always be prepared using techniques that ensure that they are free of microbial contamination. Antigen preparations may be sterilized by passage through a 0.22 um filter.

### Purification of Polyclonal Antibodies

Polyclonal and monoclonal antibodies may be purified from non-immunoglobulin contaminants using known techniques, for example, use of a protein-A or protein-G affinity chromatography column. Polyclonal antibodies in accordance with the present invention may require further purification in order to eliminate or reduce cross reactivities. In order to remove cross-reactivities to non-activated Factor XII it may be necessary to remove those species of antibody from the polyclonal sera by a process of affinity purification. Fisher et al., 1988, Cell 54: 813-822, the disclosure of which is incorporated herein by reference, gives details of a suitable protocol for affinity purification of a polyclonal antibody. In essence, such purification techniques involves immobilizing the antigen or antigen that are causing the cross-reactivity problem on a solid substrate, for example the walls of an article of laboratory plastic-ware, or solid beads packed inside a chromatography column, and passing the polyclonal sera through or over the solid substrate so that antibody species exhibiting cross reactivity are retained and antibody species that do not show cross reactivity are retained in the liquid phase. As an example of the use of an affinity purification technique for the production of a polyclonal antibody of the present invention, an polyclonal antibody response could be raised in an animal by inoculating that animal with an activated form of Factor XIIa; the resultant polyclonal sera could then be affinity purified by passing it through a chromatography column containing immobilised non-activated Factor αXIIa. Antibody species showing cross reactivity with non-activated Factor XII would be retained in the column and antibody species capable of binding to a form of activated Factor XIIa but not non-activated Factor XII would remain in the liquid phase and be contained in the column eluate.

### Samples and Sample Preparation

### Samples

Measurement of different forms of activated Factor XIIa may be performed on a sample of a body fluid, for example, whole blood, plasma, serum, urine, cerebrospinal fluid, saliva or tears; or a sample comprising cells isolated from a body fluid, that is to say, cells substantially free from the liquid phase in which they exist in vivo; or a sample comprising tissue or cells obtained from a tissue sample. Preferably, measurement is carried out on a sample of plasma.

### Sample Preparation

Samples may be obtained and prepared according to normal practice, see for example, Young, D. S. & Bermes, E. W. “Specimen collection and processing” in Tietz Textbook of Clinical Chemistry 2^{nd} Edition” Eds. Burtis, C. A. & Ashwood, E. R, Saunders (1994), also Methods in Enzymology, H. Van Vunakis and J. J. Langone (Eds), 1981, 72(B); Practice and Theory of Enzyme Immunoassays, P Tijssen, Laboratory Techniques in Biochemistry and Molecular Biology, R. J. Burden and P. H. Van Knippenberg (Eds), Elsevier, 1985; Introduction to Radioimmunoassay and Related Techniques, T. Chard, ibid, 3rd Edition, 1987; and Methods in Enzymology, H. Van Vunakis and J. J. Langone (Eds) 1981, 74(C).

### Antibody Cross Reactivity

The antibodies of the invention have corrected cross-reactivity with non-activated Factor XII of 10% or less, more preferably 5% or less, still more preferably 2% or less, still more preferably 1% or less, still more preferably 0.5% or less, still more preferably 0.1% or less. Preferably, the antibodies have a low cross reactivity, for example of 0.5% or less or more preferably of 0.1% or less with Factor XII. A factor to take into consideration in assessing the cross-reactivity of an antibody of the invention with Factor XII is that as explained in Silverberg and Kaplan, Blood 60, 1982, 64-70 preparations of Factor XII are inevitably contaminated with Factor XIIa. WO90/08835 gives details of methods of assessing the corrected cross-reactivity with Factor XII. Unless specified otherwise, the term “cross reactivity” is used herein to mean the corrected cross reactivity.

Methods used to produce monoclonal antibodies are well known, see for example, Methods in Enzymology, H. Van Vunakis and J. J. Longone (Eds) 1981, 72(B) and ibid, 1983 92(E). Monoclonal antibodies may be produced, for example, by a modification of the method of Kohler and Milstein (G. Kohler and C. Milstein, Nature, 1975, 256, 495).

WO 90/08835, which is incorporated herein by reference, describes in general terms how to produce an antibody that binds to activated Factor XII and that has shown a corrected cross-reactivity with Factor XII of 0.1% or less, and gives specific details of the production of mAb 2/215 and mAb 201/9. The general and specific methods described therein may used to produce a monoclonal antibody suitable for use according to the present invention, for example, a monoclonal antibody binding to activated Factor XIIa but not binding to unactivated Factor XII. A general protocol for producing monoclonal antibodies suitable for use according to the present invention, based on the disclosure of WO90/08835, is given in Example 22 of WO04/057343 which is incorporated herein by reference.

Methods used to produce monoclonal antibodies are well known, see for example, Methods in Enzymology, H. Van Vunakis and J. J. Longone (Eds) 1981, 72(B) and ibid, 1983 92(E). Monoclonal antibodies may be produced, for example, by a modification of the method of Kohler and Milstein (G. Kohler and C. Milstein, Nature, 1975, 256, 495). The antigen used in the production of monoclonal antibodies may be Factor αXIIa or 53 Kd Factor XIIa or Factor βXIIa. Resulting monoclonal antibodies may be screened for those that show no significant binding to Factor XII one, for example, having a corrected cross-reactivity with Factor XII of 0.1% or less.

It may be advantageous to use monoclonal antibody 2/215 or 201/9, respectively, as a reference antibody in screening for antibodies that bind to activated Factor XIIa.

The invention is not limited to hybridomas of murine or part-murine origin. Both fusion partners (spleen cells and myelomas) may be obtained from any suitable animal. Recombinant antibodies may be produced. Antibodies may be brought into chimeric or humanized form, if desired. Hybridomas are preferably cultured in vitro.

### Polyclonal Antibodies

The present invention also provides polyclonal antibodies, also called a polyclonal antiserum, that are capable of reacting selectively with one or more forms of activated Factor XIIa.

### Angioplasty

Angioplasty is a surgical technique used to treat patents with diseased arteries.

The procedure widens (dilates) blocked arteries, which can help prevent the complications of atherosclerosis. Angioplasty is usually combined with implantation of a stent in the clogged artery to help prop it open and decrease the chance of re-blockage. Angioplasty is also known as coronary artery balloon dilation, balloon angioplasty and percutaneous coronary intervention (PCI).

Angioplasty is commonly performed through an artery in the patient's groin (femoral artery). Less commonly, it may be done using an artery in the arm or wrist area.

A short tube called a sheath is inserted into the femoral artery. A guide is then inserted into the sheath. Aided by X-ray images on a monitor, the doctor threads the guide catheter through that artery all the way up until it reaches the blocked or narrowed artery.

Typically an anti-coagulant, typically heparin is administered in preparation for angioplasty. A contrast agent is also injected so that area of blockage or narrowing in the artery show up on the X-ray images, so that the doctor knows where to target treatment.

### Framingham Risk Scoring Method

The Framingham Heart Study is a long term and on-going research project of the US National Heart Lung and Blood Institute and Boston University. One of the outcomes of this study was the development of the Framingham risk score which can be used to identify individuals at risk of coronary heart disease. A risk score is calculated for an individual by adding up point allocated to that individual on the basis of factors such as age, life-style and medical test results.

The invention provides a method of preventing arterial thrombosis in a subject comprising administering to said subject a therapeutically effective amount of an antibody or epitope-binding fragment or derivative thereof, wherein said antibody, fragment or derivative specifically binds to activated Factor XIIa and prevents the interaction of activated Factor XIIa with its physiological substrates, wherein said subject has an estimated risk for Coronary heart disease of more than 10, 12, 14, 16, 18, 20, 22, 24, 26, 28 or 30% as defined using the Framingham risk scoring method.

The invention also provides similar compositions products and uses. The invention encompasses related methods wherein the estimated risk score is calculated by a variant, derivative, improvement or alternative to the Framingham risk scoring method.

The invention also encompasses similar methods for preventing any disorder caused by undesirable thrombus formation, wherein said subject has a similarly high risk of that disorder, for example a similarly high risk of stroke as defined using a suitable risk scoring method.

### Pharmaceutical Compositions

The invention relates to pharmaceutical compositions that contain an active ingredient comprising an antibody, fragment or derivative of the invention or a therapeutic agent of the invention or an in vivo imaging agent of the invention (collectively known as “active ingredient”), in combination with a pharmaceutically acceptable carrier. Typically, such pharmaceutical compositions will be in a form suitable for injection or infusion.

The compositions may conveniently be presented in unit dosage form and may be prepared by any of the methods well known in the art of pharmacy. All methods include the step of bringing the active ingredient into association with a pharmaceutical carrier which constitutes one or more accessory ingredients. In general the formulations are prepared by uniformly and intimately bringing into association the active ingredient with liquid carriers or finely divided solid carriers or both and then, if necessary, preparing the product into the desired formulation.

Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, preferably at a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition preferably does not include compounds that are known to be deleterious to the active ingredients.

Excipients that can be included are, for instance, other proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate.

Preferred unit dosage formulations are those containing an effective dose, as hereinbefore recited, or an appropriate fraction thereof, of the active ingredient.

A composition of the invention may be delivered by way of a pump (see Langer, Science 249:1527-1533, 1990; Sefton, CRC Crit. Ref. Biomed. Eng. 14:201, 1987; Buchwald et al., Surgery 88:507, 1980; Saudek et al., N. Engl. J. Med. 321:574, 1989) or by a continuous subcutaneous infusions, for example, using a mini-pump. An intravenous bag solution may also be employed. The key factor in selecting an appropriate dose is the result obtained, as measured by decreases in total body weight or ratio of fat to lean mass, or by other criteria for measuring control or prevention of obesity or prevention of obesity-related conditions, as are deemed appropriate by the practitioner. Other controlled release systems are discussed in the review by Langer, supra In another aspect of the disclosure, compounds of the invention are delivered by way of an implanted pump, described, for example, in U.S. Pat. No. 6,436,091; U.S. Pat. No. 5,939,380; U.S. Pat. No. 5,993,414.

Implantable drug infusion devices are used to provide patients with a constant and long term dosage or infusion of a drug or any other therapeutic agent. Essentially such device may be categorized as either active or passive. A compound of the present invention may be formulated as a depot preparation. Such a long acting depot formulation can be administered by implantation, for example subcutaneously or intramuscularly; or by intramuscular injection. Thus, for example, the compounds can be formulated with suitable polymeric or hydrophobic materials, for example as an emulsion in an acceptable oil; or ion exchange resins; or as a sparingly soluble derivatives, for example, as a sparingly soluble salt.

A therapeutically effective amount of a compound of the invention may be administered as a single pulse dose, as a bolus dose, or as pulse doses administered over time. Thus, in pulse doses, a bolus administration of a compound of the invention is provided, followed by a time period wherein a compound of the invention is administered to the subject, followed by a second bolus administration. In specific, non-limiting examples, pulse doses of a compound of the invention are administered during the course of a day, during the course of a week, or during the course of a month.

### Imaging Agents

The invention provides an in vivo imaging agent comprising an antibody or epitope-binding fragment or derivative thereof according to the invention attached to a marker moiety in addition to a method of imaging sites of thrombus formation in a subject comprising administering to said subject an in vivo imaging agent according to the invention followed by the detection of the imaging agent marker moiety in vivo. Such an agent and method not only have clinical uses, but can be used as a research tool, particularly in the imaging of thrombus formation in non-human animal models of thrombus formation. Imaging systems include GE Healthcare's eXplore Optix® system of fluorescence imaging which uses imaging agents attached to fluorescence markers, other imaging systems use bioluminescent markers or radioisotope markers. The present invention provides an imaging agent which will accumulate at sites of thrombus formation. This accumulation is independent of the marker moiety to which it is attached and the invention encompasses imaging agents comprising any suitable marker moiety.

### Therapeutic Agents

The invention provides a therapeutic agent comprising an antibody or epitope-binding fragment of derivate thereof according to the invention attached to a therapeutic compound, and a method of treating a disease characterised by undesirable thrombus formation in a subject comprising administering to said subject a therapeutic agent according to the invention. A number of therapeutic compounds are known for the treatment of undesirable thrombus formation in a subject. Such compounds include thrombolytic agents or “clot-busters” and also inhibitors of platelet aggregation. Specific compounds include streptokinase, urokinase, tPA, Tirofiban and Clopidogrel. By conjugating such agents to an antibody or epitope-binding fragment or derivative of the invention, therapeutic compounds can be targeted to sites of thrombus formation because such sites are likely to contain elevated levels of activated Factor XIIa Advantages of targeting therapeutic compounds to the site in which they are needed include greater therapeutic efficiency due to higher local concentrations of the therapeutic compound and lower side-effects due to lower systemic concentrations of the compound. By inhibiting the interaction of activated Factor XIIa with its physiological substrates, antibodies, fragments and derivatives thereof of the invention exhibit anti-thrombotic activity. If said antibodies are coupled to a further anti-thrombotic compound, it is likely that there will be a synergistic increase in anti-thrombotic activity.

The following non-limiting Examples illustrate the present invention.

### EXAMPLES

### Example 1

### XIIa in Restenosis Following Percutaneous Transluminal Coronary Angioplasty

Percutaneous coronary intervention (PCI) encompasses a variety of procedures used to treat patients with diseased arteries of the heart, for example, chest pain caused by a build-up of fats, cholesterol, and other substances from the blood (referred to as plaque) that can reduce blood flow to a near trickle, or a heart attack caused by a large blood clot that completely blocks the artery.

Typically, PCI is performed by threading a slender balloon-tipped tube—a catheter—from an artery in the groin to a trouble spot in an artery of the heart (this is referred to as percutaneous transluminal coronary angioplasty—also known as PTCA, coronary artery balloon dilation or balloon angioplasty). The balloon is then inflated, compressing the plaque and dilating the narrowed coronary artery so that blood can flow more easily. This is often accompanied by inserting an expandable metal stent. Stents are wire mesh tubes used to prop open arteries after PTCA.

Restenosis of the artery following PCI is a major limitation of the technique, and means of identifying and specifically treating those patients at risk would be extremely useful.

Eleven patients undergoing elective PCI to at least one lesion, had activated Factor XII levels measured in blood samples taken immediately before the start of the procedure (before administration of heparin and contrast fluid) and after the administration of heparin and contrast fluid.

All patients were taking 300 mg aspirin in advance of the procedure and were given 300 mg clopidogrel either prior to or just after PCI. Unfractionated heparin was administered intravenously to all patients before PCI [5000-7500 IU bolus, adjusted to achieve an ACT (activated clotting time)>300 seconds] immediately prior to the procedure. After local anesthesia, a femoral or radial artery sheath was placed by a single-wall entry technique.

After local anesthesia, a femoral artery sheath was placed by a single-wall entry technique. Coronary angiography was performed with standard technique in all patients, using a non-ionic contrast medium (Iomeron-R) introduced via the catheters, applying several injections throughout the procedure. Routine PCI was performed with monorail balloon catheters. Blood samples were obtained from the arterial sheath.

Blood samples were centrifuged for 15 min at 2000-×g at 20° C. Measurement of XIIa was performed immediately following centrifugation.

XIIa measurements were performed on all samples using a microtitre plate ELISA assay. The assay employed a highly specific monoclonal antibody (Mab 2/215) that reacts with XIIa but shows no detectable binding to Factor XII zymogen.

Factor XIIa was measured using microtitre plates coated with Mab 2/215 at 15 ug/ml. The sample incubation step was performed in the absence of an agent such as Triton, which has the capability of releasing XIIa that is otherwise unavailable for binding to the antibody. Thus this assay only measured XIIa where the binding of the antibody to the epitope on XIIa was not sterically hindered by it being complexed to other species. The antibody used as the conjugate was a polyclonal antibody raised against Factor XII zymogen. This polyclonal antibody reacts with multiple epitopes, but of particular importance this antibody binds to regions of the heavy non-catalytic chain of βXIIa.

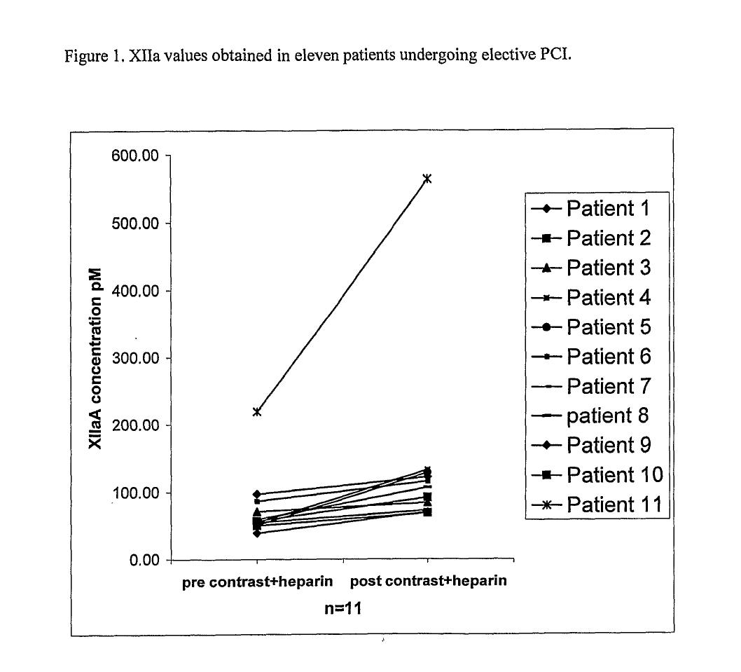

Factor XIIa values obtained in these 11 patients are shown in Table 1 and in FIG. 1. Ten of the eleven patients showed no evidence of restenosis following PCI, whereas one patient (patient 11) had restenosis (requiring treatment by emergency thrombolysis) 3 days following PCI. This patient had a markedly higher XIIa concentration both pre and post infusion with heparin/contrast media compared to the other 10 patients. These data suggest that the elevated XIIa before the physical intervention of the PCI procedure commenced demonstrated that this patient was at risk of restenosis. It is postulated that the restenosis was a result of the elevated XIIa concentration, and that treatment with XIIa inhibitors would reduce the likelihood of restenosis occurring.

TABLE 1
XIIa values obtained in eleven patients undergoing elective PCI.

| | FXIIa Concentration | FXIIa Concentration |
|---|---|---|
| | (pM) pre-heparin | (pM) post-heparin |
| Patient | and contrast media | and contrast media |
| 1 | 97.14 | 121.70 |
| 2 | 57.40 | 91.55 |
| 3 | 71.13 | 83.89 |
| 4 | 54.87 | 132.27 |
| 5 | 51.03 | 128.00 |
| 6 | 86.75 | 115.39 |
| 7 | 54.75 | 72.77 |
| 8 | 59.62 | 106.29 |
| 9 | 39.38 | 70.12 |
| 10 | 50.57 | 68.65 |
| 11 | 219.15 | 562.84 |

### Example 2

This example demonstrates that elevated levels of Factor XIIa provides are associated with increased risk of all cause mortality in patients admitted to hospital with suspected myocardial infarction and acute coronary syndrome.

Data was obtained on 871 patients admitted to the hospital. Each patient had Factor XIIa measured. Data from these assays were studied to ascertain if it provided prediction of the primary clinical endpoints of all cause mortality.

The prognostic utility of the assays was determined by ranking the Factor XIIa values (from lowest to highest) and then splitting the population into quartiles i.e. the 25% of individuals with the lowest Factor XIIa concentrations were in the 1^{st} quartile, whilst the 25% of individuals with the highest concentrations were in the 4^{th} quartile.

The form of XIIa was measured using high performance liquid chromatography following reaction of the sample with Iodine 125 labelled antibody.

Fab antibody fragments of antibody 2/215 were prepared using an “Immunopure Fab Preparation Kit” (Pierce, 3747 N Meridian Road, PO Box 117, Rockford, Ill. 61105, U.S.A.) according to manufacturers instructions. These Fab fragments were then radiolabelled with Iodine 125 by Amersham Pharmacia Biotech (Pollards Wood, Nightingales Lane, Chalfont St Giles, HP8 4SP United Kingdom).

1 μl of radiolabelled antibody was added to 1 ml of plasma from each of a number of healthy volunteers. After incubation for 4 hours, the components of the plasma were separated by High Performance Liquid Chromatography (HPLC). The HPLC system was an Agilent 1100 system.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min⁻¹ and the injection volume was 100 μl.

The HPLC eluant was monitored by measuring the absorbance at 280 nm, and by monitoring radioactivity using a Flow-Count Radiochromatography detector (LabLogic, Sheffield, UK)

Molecular weight standards were run, and from comparison with these the XIIa peaks could be identified. Integration of the area under this peak (radioactivity signal) provided a quantitative measure of XIIa. Calibration of quantitation was obtained by running standards with known quantities of the 30 kD form of XIIa (βXIIa).

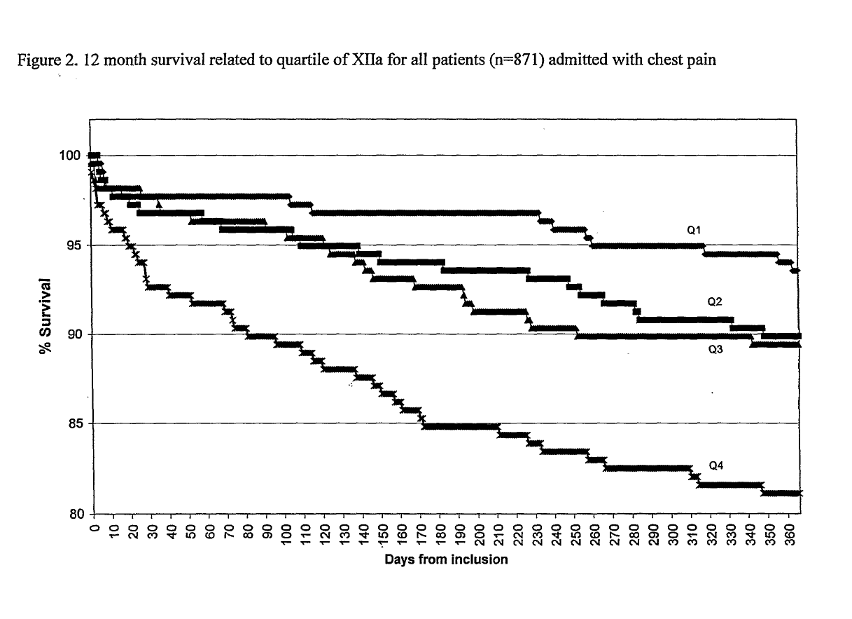

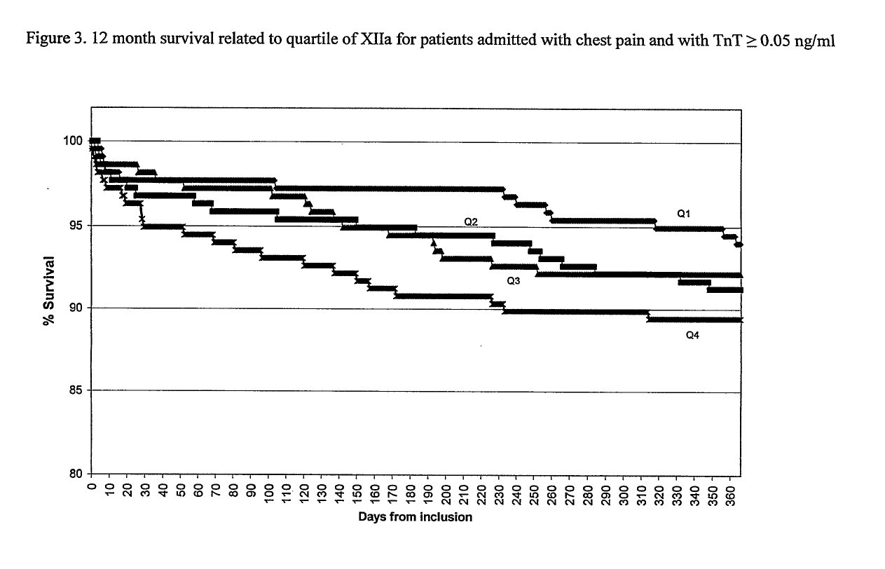

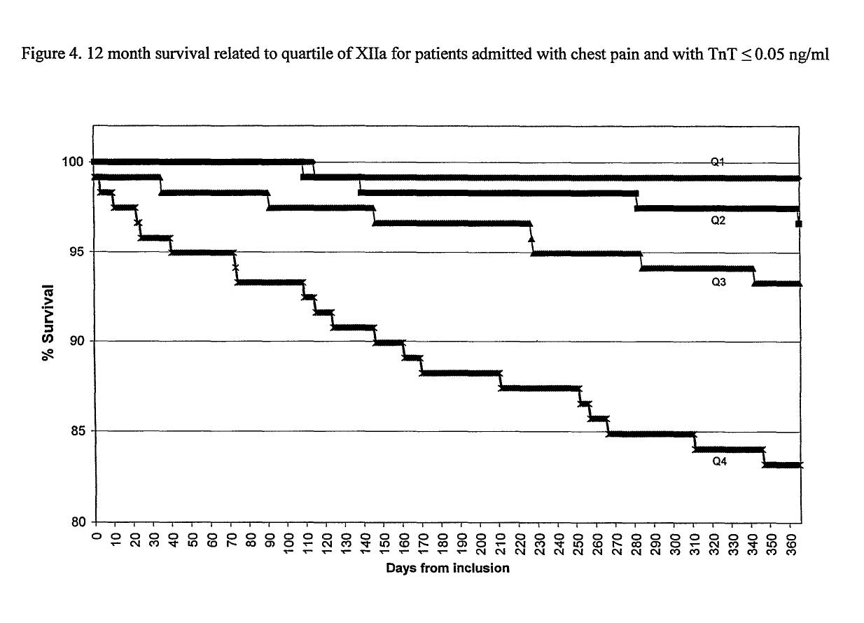

Table 2 shows the relative risk of all cause mortality related to the concentration of XIIa at different follow-up timepoints. In all cases those patients with the highest XIIa concentration were at statistically significant increased risk of death. This was true for all patients, patients admitted with myocardial infarction (defined as admission Troponin T (TnT) greater than 0.05 mg/ml but particularly in patients admitted with Troponin negative (TnT less than or equal to 0.05 ng/ml) chest pain. FIGS. 2 to 4 show Kaplan Meier survival plots for all patients, patients who had admission TnT greater than 0.05 ng/ml and patients who had admission TnT less than or equal to 0.05 ng/ml respectively.

TABLE 2
Odds ratios for all cause mortality pertaining to XIIa concentration.

| 53 kD Factor XIIa | | Q1 | Q2 | Q3 | Q4 |
|---|---|---|---|---|---|
| quartile (Range pM) | | (<25.0) | (25.0-35.0) | (35.1-55.0) | (>55.0) |
| 30 days | all patients | 1.00 | 1.68 | 1.52 | 4.34** |
| | TnT ≦ | 1.00 | 1.00 | 3.12 | 16.1** |
| | 0.05 ng/mL | | | | |
| | TnT > | 1.00 | 1.33 | 0.88 | 2.45* |
| | 0.05 ng/mL | | | | |
| 6 months | all patients | 1.00 | 2.09 | 2.39* | 5.38** |
| | TnT ≦ | 1.00 | 2.12 | 4.10 | 15.7** |
| | 0.05 ng/mL | | | | |
| | TnT > | 1.00 | 1.84 | 2.31 | 3.92** |
| | 0.05 ng/mL | | | | |
| 12 months | all patients | 1.00 | 1.64 | 1.82 | 3.93** |
| | TnT ≦ | 1.00 | 4.30 | 7.95* | 24.98** |
| | 0.05 ng/mL | | | | |
| | TnT > | 1.00 | 1.62 | 1.64 | 2.10* |
| | 0.05 ng/mL | | | | |

*p < 0.05
**p < 0.01

### Example 3

This example demonstrates that changes in concentration of Factor XIIa provides are associated with risk of secondary myocardial infarction in patients admitted to hospital with myocardial infarction.

Data was obtained on 315 patients admitted to the hospital. Blood samples were obtained at admission and 4 days after admission. Each patient had Factor XIIa measured. Data from these assays were studied to ascertain if changes in the concentration of Factor XIIa provided prediction of the primary clinical endpoints of a second myocardial infarction within 30 days of admission. At 30 days follow-up, 24 patients had suffered a secondary myocardial infarction.

XIIa was measured using high performance liquid chromatography following reaction of the sample with Iodine 125 labelled antibody.

Fab antibody fragments of antibody 2/215 were prepared using an “Immunopure Fab Preparation Kit” Pierce, 3747 N Meridian Road, PO Box 117, Rockford, Ill. 61105, U.S.A.) according to manufacturers instructions. These Fab fragments were then radiolabelled with Iodine 125 by Amersham Pharmacia Biotech (Pollards Wood, Nightingales Lane, Chalfont St Giles, HP8 4SP United Kingdom).

1 μl of radiolabelled antibody was added to 1 ml of plasma from each of a number of healthy volunteers. After incubation for 4 hours, the components of the plasma were separated by High Performance Liquid Chromatography (HPLC). The HPLC system was an Agilent 1100 system.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min⁻¹ and the injection volume was 100 μl.

The HPLC eluant was monitored by measuring the absorbance at 280 nm, and by monitoring radioactivity using a Flow-Count Radiochromatography detector (LabLogic, Sheffield, UK) Molecular weight standards were run, and from comparison with these the XIIa peaks could be identified. Integration of the area under these peaks (radioactivity signal) provided a quantitative measure of XIIa. Calibration of quantitation was obtained by running standards with known quantities of the 30 kd form of XIIa (βXIIa).

The prognostic utility of the assays was determined by ranking the change in Factor XIIa values (from lowest to highest) and then splitting the population into quartiles i.e. the 25% of individuals with the greatest decrease in Factor XIIa concentrations between admission and day 4 were in the 1^{st} quartile, whilst the 25% of individuals with the greatest increase in concentrations were in the 4^{th} quartile.

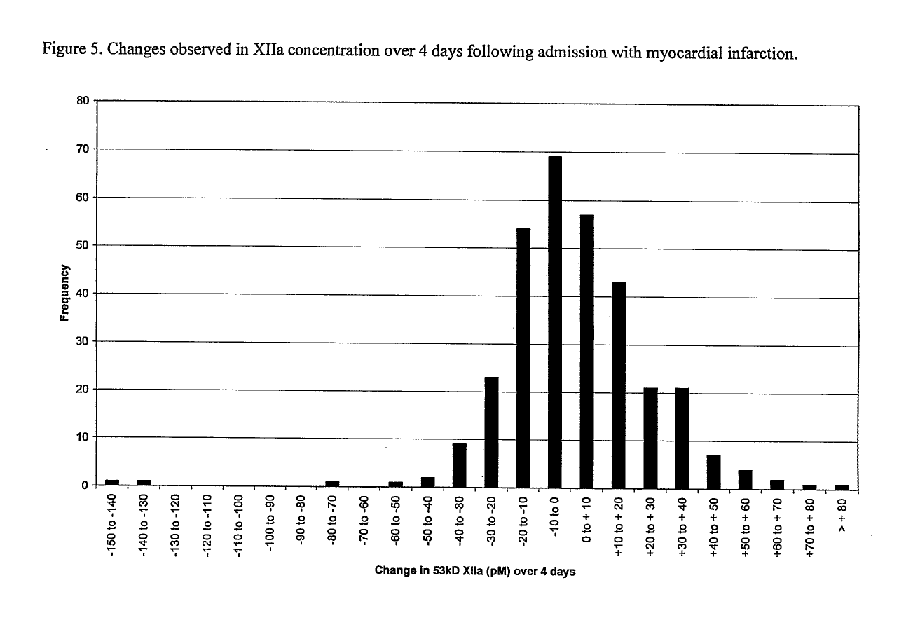

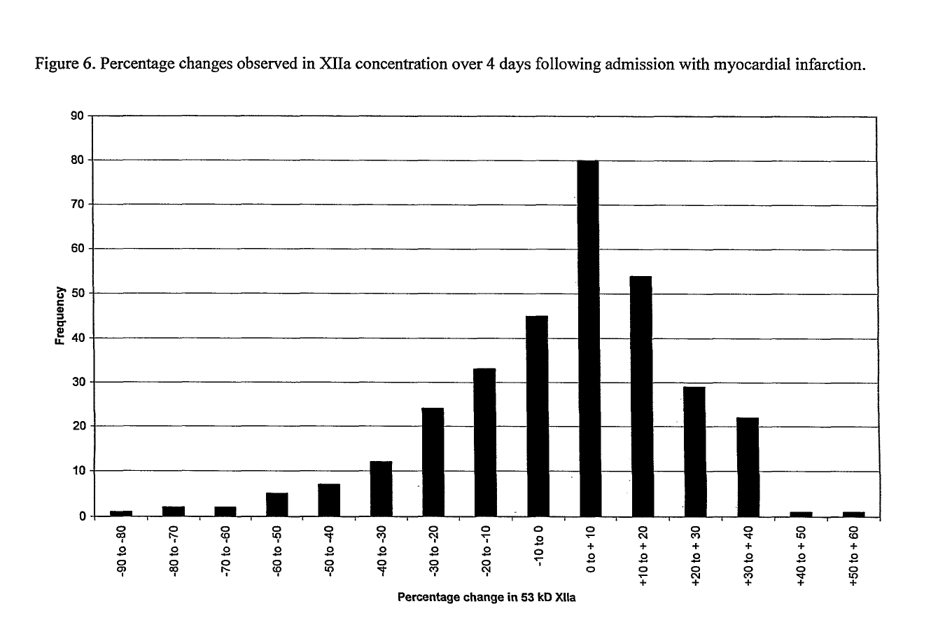

The distribution of changes in the concentration of XIIa (expressed as pM) are shown in FIG. 5, and the relative changes in the concentration of the 53 kD form of XIIa (expressed as percentage change relative to the admission value) are shown in FIG. 6.

Event-rates according to change in XIIa concentration are given in table 3. Both absolute and relative (percentage change from admission) changes in XIIa concentration were strongly associated with risk. The odds ratio for recurrent TnT positive events in Q4 as compared to Q1 of change in XIIaA concentration was 15.36 (p=0.0046) for absolute change and 13.97 (p=0.0062) for percentage change relative to the admission value. Therefore, it is concluded that changes in XIIa concentration from admission to day 4 after myocardial infarction strongly predict myocardial infarction during 30 days follow-up.

TABLE 3
Incidence of TnT positive cardiac events within 30 days following
hospitalisation for MI, related to change in XIIa between
admission and 4 days post MI.

| | Q1 | Q2 | Q3 | Q4 |
|---|---|---|---|---|
| Change in (pM) | | | | |
| Recurrent TnT + | 1 | 4 | 6 | 13 |
| events (n) | | | | |
| OR (p) | 1.0 | 4.16 (0.104) | 6.41 (0.044) | 15.36 (0.0046) |
| Change in 53 kD XIIa | | | | |
| (expressed as % | | | | |
| of admission | | | | |
| value) | | | | |
| Recurrent TnT + | 1 | 4 | 7 | 12 |
| events (n) | | | | |
| OR (p) | 1.0 | 4.16 (0.104) | 7.58 (0.030) | 13.97 (0.0062) |

### Example 4

This example demonstrates the presence of activated Factor XII as a constituent of arterial thromboses by immunohistochemical staining, illustrating the involvement of activated Factor XII in thrombosis formation.

Samples of thrombotic material was obtained from cardiac arteries of individuals undergoing acute percutaneous transluminal coronary angioplasty at a specialist regional centre in Norway. A range of other tissues were also obtained to act as suitable controls for the detection of activated Factor XII. Ethical committee approval and informed patient consent was provided for the study.

Tissue samples (including thrombotic material) were examined for the presence of Factor XIIa using antibodies specific for this molecule in conjunction with the Dako Envision immunohistochemistry system.

Prior to immunohistochemistry staining, tissues were fixed and processed. Fixation prevents autolysis and necrosis of excised tissue and preserves the antigenicity of the sample. Samples were fixed using 10% neutral phosphate buffered formalin. Following fixation, processing was completed using an automated tissue processor. Tissues were dehydrated using graded alcohol solution, cleared with xylene and infiltrated with paraffin wax. The tissue was then embedded with paraffin wax in a cassette.

Embedded tissue was sectioned using a microtome and sectioned tissues were collected on clean glass slides. Samples were dehydrated by incubation in an oven at 56° C. for 60 minutes. Prior to staining, tissue slides were deparaffinised by placing the slides in a xylene bath and incubating for 5 minutes. Slides were then placed in a second fresh xylene batch and incubated for a further 5 minutes. Excess fluid was tapped off slides and these were then placed in an absolute alcohol bath for 3 minutes. Excess fluid was tapped off and slides were placed in a second bath containing fresh absolute alcohol. Excess fluid was tapped off slides and these were then placed in a 95% alcohol bath for 3 minutes. Excess fluid was tapped off and slides were placed in a second bath containing fresh 95% alcohol. Excess liquid was tapped off and slides were placed in distilled water for 60 seconds. Slides were then stored in 0.05M Tris buffered saline pH 7.4 until the next stage of processing.

Excess buffer was tapped off the slide upon which the tissue section was mounted. Any remaining liquid was removed by carefully wiping a lintless tissue around the specimen. The specimen was covered with the Dakocytomation Peroxidase Block reagent and was incubated for 5 minutes.

The tissue sample was then gently rinsed with 0.05M Tris buffered saline from a wash bottle, and placed in a fresh buffer bath containing 0.05M Tris buffered saline.

At the start of the next step, excess buffer was tapped off the slide upon which the tissue section was mounted. Any remaining liquid was removed by carefully wiping a lintless tissue around the specimen. The specimen was covered with 10 ug/ml 2/215 monoclonal antibody in 0.05 M Tris-HCl, pH 7.4 containing 1% bovine serum albumin (DakoCytomation Envision Antibody Diluent, code S0809) and incubated for 30 minutes.

Duplicate samples were incubated with a control antibody in place of the 2/215 to act as a control antibody. The negative control antibody was a murine monoclonal antibody (same subclass as 2/215) directed against hamster IgG (Sigma H2412), and concentrations and incubation times were identical to those used for the 2/215 anti-XIIa antibody. Additional negative controls were run using the DakoCytomation Universal Negative Control (code NP015).

Following the 30 minute incubation with primary antibodies, The tissue samples were gently rinsed with 0.05M Tris buffered saline from a wash bottle, and placed in a fresh buffer bath containing 0.05M Tris buffered saline.

At the start of the next step, excess buffer was tapped off the slide upon which the tissue section was mounted. Any remaining liquid was removed by carefully wiping a lintless tissue around the specimen. Specimens were then covered with the DakoCytomation Envision labeled polymer and incubated for 30 minutes. The tissue samples were gently rinsed with 0.05M Tris buffered saline from a wash bottle, and placed in a fresh buffer bath containing 0.05M Tris buffered saline.

Excess buffer was tapped off the slide upon which the tissue section was mounted. Any remaining liquid was removed by carefully wiping a lintless tissue around the specimen. The specimens were then covered with DakoCytomation Envision liquid DAB+ substrate-chromagen solution that had been reconstituted according to manufacturers instructions and incubated for 10 minutes. Samples were then gently rinsed with distilled water from a wash bottle.

Samples were then counterstained by immersion in a bath of haematoxylin for 2 minutes, followed by gentle rinsing in a distilled water bath. Slides were then dipped into a batch containing 0.037M ammonia, and then rinsed in a batch of distilled water for 5 minutes. Specimens were then mounted and coverslipped using nonaqueous permanent mounting media.

Specimens were then examined microscopically for the presence of immunohistochemical (IHC) staining (evident as a brown stain), and the results are summarised in table 4.

As a considerable degree of IHC staining was evident in the thrombus material when using monoclonal antibody 2/215 (anti-Factor XIIa) but no staining in this tissue was evident with the control antibodies, it can be included that Factor XIIa is a significant constituent of the arterial thrombus. The extensive staining observed indicates that the activated Factor XII is present in high concentration, and it therefore reasonable to conclude that the activated Factor XII plays an important role in thrombus formation and/or stabilization.

TABLE 4
Results from immunohistochemical staining of different tissue samples
with 2/215 anti Factor XIIa and negative control antibodies.

| | | Universal Negative | |
|---|---|---|---|
| Tissue | Anti-Hamster IgG | Control | 2/215 anti XIIa |
| Uterus | No IHC staining | No IHC staining | No IHC staining evident (no |
| | evident | evident | granulocytes observed within tissue) |
| Placenta | No IHC staining | No IHC staining | IHC Staining of granulocytes present |
| | evident | evident | within the tissue. |
| Tonsil | No IHC staining | No IHC staining | IHC Staining of granulocytes present |
| | evident | evident | within the tissue. |
| Arterial | No IHC staining | No IHC staining | Extensive strong IHC staining of |
| Thrombus | evident | evident | thrombus material. |

### Example 5

This example demonstrates the presence of activated Factor XII as a constituent of arterial thromboses by ELISA of homogenised tissue, illustrating the involvement of activated Factor XII in thrombus formation.

Samples of thrombotic material was obtained from cardiac arteries of individuals undergoing acute percutaneous transluminal coronary angioplasty at a specialist regional centre in Norway. Ethical committee approval and informed patient consent was provided for the study.

A 92 mg sample of arterial thrombus was homogenised using an Omni hard tissue tip homogenising kit, the final volume of the homogenate being 920 ul

The tissue homogenate underwent serial dilution, such that the total tissue weight/volume content of each dilution was 100 mg/ml, 10 mg/ml, 1 mg/ml and 100 ug/ml.

Monoclonal antibody 2/215 was coated on a Nunc (Nunc A/S, Karustrupuej 90, P O Box 280, 4000 Roskilde, Denmark) Maxisorb microplate (100 μl of antibody was coated per well) at a concentration of 15 μg ml⁻¹ in a carbonate coating buffer pH. 9.6). 100 μl of each tissue homogenate dilution with Triton X-100 (Sigma, Fancy Road, Poole, Dorset, England) added to a final Triton concentration of 0.5% (v/v) was added to the wells of the microtitre plate and incubated for 60 minutes at room temperature. After washing the wells of the microtitre plate, 100 μl of conjugate was added. This conjugate comprised monoclonal antibody 201/9 conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5) and the absorbance at 550 nm was measured. Standards of βXIIa (0, 1, 2.5, 5 & 10 ng/ml) were also run in the assay. Results are shown in Table 5. All of the thrombus homogenate dilutions gave an absorbance above that of the top βXIIa standard indicating that the thrombus contains a high concentration of activated Factor XII.

TABLE 5
Absorbances from ELISA of homogenised arterial thombus.

| Standard/Sample | Absorbance |
|---|---|
| 0 ng/ml βXIIa | 0.032 |
| 1.0 ng/ml βXIIa | 0.414 |
| 2.5 ng/ml βXIIa | 0.832 |
| 5.0 ng/ml βXIIa | 1.201 |
| 10.0 ng/ml βXIIa | 1.623 |
| 100 mg/ml homogenised thrombus | >2.0 |
| 10 mg/ml homogenised thrombus | >2.0 |
| 1 mg/ml homogenised thrombus | >2.0 |
| 100 ug/ml homogenised thrombus | >2.0 |

What is claimed is:

1. A method of preventing arterial thrombosis in a subject comprising administering to said subject a therapeutically effective amount of an antibody or epitope-binding fragment or derivative thereof, wherein said antibody, fragment or derivative specifically binds to activated Factor XIIa and prevents the interaction of activated Factor XIIa with its physiological substrates.

2. A method as claimed in claim 1, wherein said antibody or epitope-binding fragment or derivative thereof binds to Factor αXIIa or to Factor βXIIa or to 53 Kd factor XIIa, and has a corrected cross-reactivity with un-activated Factor XII of 0.1% or less.

3. A method of preventing arterial thrombosis in a subject comprising administering to said subject a therapeutically effective amount of an antibody or epitope-binding fragment or derivative thereof, wherein said antibody or epitope-binding fragment or derivative thereof prevents multi molecular assemblies of Factor XII or wherein said antibody or epitope-binding fragment or derivative thereof prevents formation of activated Factor XIIa.

4. A method as claimed in any of claims 1 to 3, wherein said administration is following angioplasty carried out on said subject.

5. A method as claimed in any of claims 1 to 3, wherein said administration is following myocardial infarction in said subject.

6. A method as claimed claim 1, wherein the subject has an estimated risk for Coronary heart disease of more than 10% as defined using the Framingham risk scoring method.

7. A method as claimed claim 1, wherein said subject has a plasma concentration of activated factor XIIa of significantly different to that of a reference population as measured before administration of said antibody or epitope-binding factor or derivative thereof.

8. A method as claimed claim 1, wherein said subject's plasma concentration of activated Factor XIIa increases by a factor significantly different to that of a reference population following administration to said subject of heparin and contrast agent in preparation for angioplasty.

9. A method as claimed in any of claims 1 to 3, wherein said antibody or epitope-binding fragment or derivative thereof is a monoclonal antibody or epitope-binding fragment or derivative thereof.

10. A method as claimed in claim 9, wherein said monoclonal antibody or epitope-binding fragment or derivative thereof is mAb 2/215 (ECACC deposit number 04061403) or an analogue thereof or mAb 201/9 (ECACC deposit number 04061402) or an analogue thereof, or an epitope-binding fragment or derivative of mAb 2/215 or an analogue thereof, or an epitope-binding fragment or derivative or of mAb 201/9 or an analogue thereof.

11. A method as claimed in any of claims 1 to 3, wherein said antibody or epitope-binding fragment or derivative thereof is a Fab fragment or a (Fab′)₂ fragment.

12. A method as claimed in any of claims 1 to 3, wherein said antibody or epitope-binding fragment or derivative thereof is a humanised antibody or epitope-binding fragment or derivative thereof.

13. An antibody or epitope-binding fragment or derivative thereof, wherein said antibody specifically binds to activated Factor XIIa and prevents the interaction of activated Factor XIIa with its physiological substrates for use as a medicament.

14. An antibody or epitope-binding fragment or derivative thereof as claimed in claim 13, wherein said antibody or epitope-binding fragment or derivative thereof is capable of binding to Factor αXIIa and or to Factor βXIIa or to 53 Kd Factor XIIa, and has a corrected cross-reactivity with un-activated Factor XII of 0.1% or less.

15. An antibody or epitope-binding fragment or derivative thereof against non-activated Factor XII for use in preventing arterial thrombosis in a subject, wherein said antibody or epitope-binding fragment or derivative thereof prevents multi molecular assemblies of Factor XII or wherein said antibody or epitope-binding fragment or derivative thereof prevents formation of activated Factor XIIa.

16. An antibody or epitope-binding fragment or derivative thereof as claimed in one of claims 13 to 15, wherein said antibody or epitope-binding fragment or derivative thereof is a monoclonal antibody or epitope-binding fragment or derivative thereof.

17. An antibody or epitope-binding fragment or derivative thereof as claimed in claim 16, wherein said antibody or epitope-binding fragment or derivative thereof is mAb 2/215 (ECACC deposit number 04061403) or an analogue thereof or mAb 201/9 (ECACC deposit number 04061402) or an analogue thereof or an epitope-binding fragment or derivative of mAb 2/215 or an analogue thereof or an epitope-binding fragment or derivative of mAb 201/9 or an analogue thereof.

18. An antibody or epitope-binding fragment or derivate thereof as claimed in claim 13, which is a Fab fragment or a (Fab)₂ fragment.

19. An antibody or epitope-binding fragment or derivative thereof as claimed in claim 13, which is a humanised antibody or epitope-binding fragment or derivative thereof.

20. A pharmaceutical composition comprising an antibody or epitope-binding fragment or derivative thereof as defined in claim 13, together with a pharmaceutically acceptable carrier.

21. (canceled)

22. (canceled)

23. (canceled)

24. (canceled)

25. A method of predicting the risk of subsequent restenosis in a subject elected to undergo percutaneous coronary intervention comprising measuring the level of activated Factor XIIa in the blood of said subject and assigning said subject to a high risk group if the subject's plasma concentration of activated factor XIIa is significantly different to that of a reference population or if the subject's plasma concentration of activated Factor XIIa increases by a factor significantly different to that of a reference population following administration to said subject of heparin and contrast agent in preparation for angioplasty.

26. An in vivo imaging agent comprising an antibody or epitope-binding fragment or derivative thereof as defined in claim 13 attached to a marker moiety.

27. An in vivo imaging agent as claimed in claim 26 wherein said marker moiety is a radiolabel or a fluorochrome.

28. A method of imaging sites of thrombus formation in a subject comprising administering to said subject an in vivo imaging agent as claimed in claim 26, followed by the detection of the imaging agent marker moiety in vivo.

29. A therapeutic agent comprising an antibody or epitope-binding fragment of derivate thereof as defined in claim 13, attached to a therapeutic compound.

30. A therapeutic agent as claimed in claim 29, wherein said therapeutic compound is a thrombolytic agent.

31. A therapeutic agent as claimed in claim 29, wherein said therapeutic compound is an inhibitor of platelet aggregation.

32. A therapeutic agent as claimed in claim 29, wherein said therapeutic compound is streptokinase, urokinase, tissue plasminogen activator (tPA), Tirofiban, Clopidogrel or Tenecteplase.

33. A method of treating a disease characterised by undesirable thrombus formation in a subject comprising administering to said subject a therapeutic agent as defined in claim 29.
